(19) United States
(12) Patent Application Publication
(10) Pub. No.: US 2026/0317669 A1
(43) Pub. Date: Oct. 15, 2026

(54) MOAT-TYPE MICROWELL CULTURE PLATE

(71) Applicant: Suzhou Cellatopia Biotechnologies Co., Ltd., Suzhou (CN)

(72) Inventor: Jingyu Li, Suzhou (CN)

(73) Assignee: Suzhou Cellatopia Biotechnologies Co., Ltd., Suzhou (CN)

(21) Appl. No.: 19/684,984

(22) Filed: May 21, 2026

Related U.S. Application Data
(63) Continuation of application No. PCT/CN2025/073818, filed on Jan. 22, 2025.

(30) Foreign Application Priority Data
Nov. 23, 2023 (CN) 202323168872.2

(51) Int. Cl. C12M 1/32

(52) U.S. Cl. CPC C12M 23/12

(57) ABSTRACT

The present application relates to the technical field of in vitro cell culture, and in particular, to a moat-type microwell culture plate that includes a microwell culture plate body. The microwell culture plate body is provided with a water injection groove. A plurality of micro culture wells are arranged inside the water injection groove, with openings of the culture wells facing upward. A water level in the water injection groove is lower than the openings of the culture wells, and water in the water injection groove surrounds the culture wells to form a structure resembling a “moat”. After the water evaporates, water vapor is produced, thereby providing a humidifying effect. Moreover, such a humidification manner is spontaneous and does not introduce bacteria, ensuring that the culture wells are not contaminated during incubation.

### CROSS-REFERENCE TO RELATED APPLICATIONS

The application claims priority to Chinese patent application No. 202323168872.2, filed on Nov. 23, 2023, the entire contents of which are incorporated herein by reference.

### TECHNICAL FIELD

The present application relates to the technical field of in vitro cell culture, and in particular, to a moat-type microwell culture plate.

### BACKGROUND

Microwell culture plates are important consumables for high-throughput in vitro culture of a small quantity of cells to facilitate subsequent high-throughput testing and research, and are mainly used for in vitro cell culture.

Due to a small volume of culture wells in a microwell culture plate, the wells can contain a very small amount of culture solution, which is extremely prone to rapid evaporation in a constant-temperature incubator, thereby altering the state of the cultured cells and affecting experimental results. Existing microwell culture plates generally do not have a built-in humidification method. Instead, they rely on external humidification methods (e.g., placing a water pan or an atomizer at the bottom of an incubator) to humidify the culture space. Although the method of using the water pan to humidify the interior of the incubator is simple to operate, the water pan, when left static in a warm environment for a long time, easily breeds bacteria or mold, leading to contamination of the culture environment. Moreover, since this method relies on natural evaporation, it is difficult to ensure uniform humidity throughout the incubator, resulting in different microenvironments for cells located in different parts of the incubator, which will ultimately cause poor stability of experimental results. The method of using a humidifier to humidify the incubator mainly involves using ultrasonic or a vapor generator to produce water mist and then dispersing the water mist throughout the incubator by a fan. Although this method allows for humidity adjustment, it tends to produce excessive moisture that causes the incubator to be difficult to clean, easily leading to microbial reproduction and contamination.

### SUMMARY

The technical problem to be solved in the present application is to provide a moat-type microwell culture plate. Culture wells are provided in a water injection groove, thereby improving the thermal insulation performance of the culture plate by means of the high specific heat capacity of water while maintaining microenvironment humidity. This solves both the problems of instability and high contamination risk associated with external humidification of existing microwell culture plates, and the problem of too rapid cooling of microwell culture plates during operation in a non-incubator environment.

To solve the technical problem described above, the following technical solution is provided.

The moat-type microwell culture plate of the present application includes a microwell culture plate body, where the microwell culture plate body is provided with a water injection groove; and micro culture wells are arranged inside the water injection groove, with openings of the culture wells facing upward.

By adopting the technical solution described above, a water level in the water injection groove is lower than the openings of the culture wells, and water in the water injection groove surrounds the culture wells to form a structure resembling a “moat”. After the water evaporates, water vapor is produced, thereby providing a humidifying effect. Moreover, such a humidification manner is spontaneous and does not introduce bacteria, such that the culture wells are not contaminated during cell incubation.

The water injection groove and the culture wells are of an integrated structure.

By adopting the technical solution described above, the effectiveness of the microwell culture plate is ensured, and the cross contamination between the water in the water injection groove and cells in the culture wells is avoided.

The culture wells arranged inside the water injection groove have equal transverse and longitudinal spacings, and both the transverse and longitudinal spacing between the culture wells are 9 mm.

By adopting the technical solution described above, the moat-type microwell culture plate can be adapted to fit standardized multi-channel pipettes and automatic pipetting devices.

The microwell culture plate body is surrounded by a rim; an annular groove is formed in a bottom of the rim; an inner edge of the annular groove is integrally connected to the microwell culture plate body; and a reinforcing rib connected to the microwell culture plate body is disposed in the annular groove.

By adopting the technical solution described above, the rim around the bottom of the microwell culture plate body is hollowed out so as to save material, and the reinforcing rib is utilized to improve the connection strength between the microwell culture plate body and the rim.

By adopting the solution described above, the present application has the following advantages.
1. In this solution, the water level in the water injection groove is lower than the openings of the culture wells, and water in the water injection groove surrounds the culture wells to form a structure resembling a “moat”. After the water evaporates, water vapor is produced, thereby providing a humidifying effect. Moreover, such a humidification manner is spontaneous and does not introduce bacteria, such that the culture wells are not contaminated during incubation.
2. In this solution, each culture well is surrounded by water in the water injection groove. The high specific heat capacity of water improves the constant temperature performance of the entire culture plate, thereby slowing the rate of cooling when the culture plate is handled outside the incubator and thus being more conducive to cell growth in the culture wells.
3. In this solution, the microwell culture plate has an integrated structure, ensuring the effectiveness of the microwell culture plate and avoiding cross contamination between the cells in the culture wells and the water in the water injection groove.

### BRIEF DESCRIPTION OF DRAWINGS

FIG. 1 is an overall schematic structural diagram of a moat-type microwell culture plate according to the present application;

FIG. 2 is a top schematic structural diagram of a moat-type microwell culture plate according to the present application; and

FIG. 3 is an overall schematic structural diagram of a moat-type microwell culture plate according to the present application.

List of Reference Numerals: 1—microwell culture plate body; 2—water injection groove; 3—micro culture well; 4—annular groove; and 5—rim.

### DETAILED DESCRIPTION OF THE EMBODIMENTS

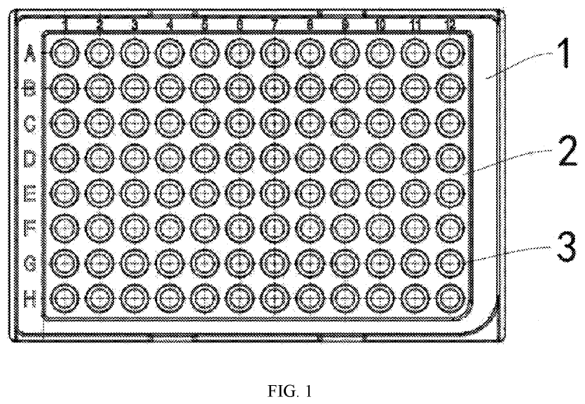

The present disclosure is described below with reference to FIG. 1 to FIG. 3 and embodiments.

In the embodiments, a moat-type microwell culture plate of this solution, as shown in FIG. 1, includes a microwell culture plate body 1. The microwell culture plate body 1 is provided with a water injection groove 2. A plurality of culture wells 3 are arranged inside the water injection groove 2, with openings of the culture wells 3 facing upward. When water is injected into the water injection groove 2, a water level in the water injection groove 2 is lower than the openings of the culture wells 3, so that the water in the water injection groove 2 surrounds the culture wells 3 to form a structure resembling a “moat”. With such a structure, after the water evaporates, water vapor is produced, which then spreads into the culture wells 3 to provide a humidifying effect. Moreover, the humidification occurs spontaneously rather than by artificial spraying. Therefore, as long as the water in the water injection groove 2 is sufficiently clean, no bacteria will be introduced, ensuring that the culture wells 3 are not contaminated during incubation.

To ensure the overall effectiveness of the microwell culture plate, the water injection groove 2 and the culture wells 3 are of an integrated structure, thereby avoiding cross contamination between cells in the culture wells 3 and the water in the water injection groove 2.

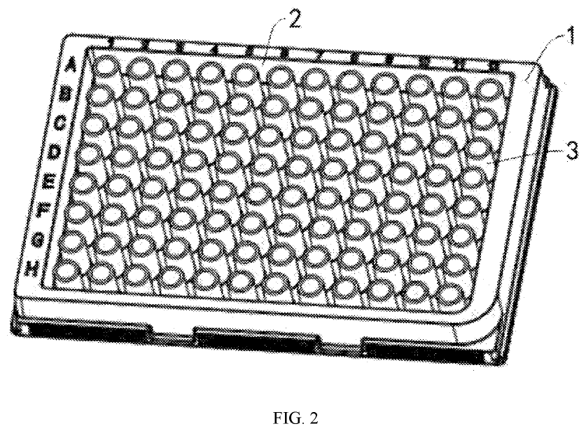

As shown in FIG. 2, to fit standardized multi-channel pipettes and automatic pipetting devices, the culture wells 3 arranged inside the water injection groove 2 have equal transverse and longitudinal spacings, and both the transverse and longitudinal spacing between the culture wells 3 are 9 mm.

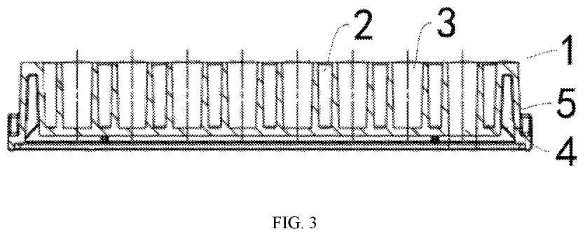

Further, as shown in FIG. 3, the microwell culture plate body 1 is surrounded by a rim 5; an annular groove 4 is formed in a bottom of the rim 5; an inner edge of the annular groove 4 is integrally connected to the microwell culture plate body 1; and a reinforcing rib connected to the microwell culture plate body is disposed in the annular groove 4. Since the rim 5 around the bottom of the microwell culture plate body 1 is hollowed out so as to save material, the reinforcing rib is utilized to improve the connection strength between the microwell culture plate body 1 and the rim 5.

The present disclosure has been described above in combination with specific embodiments. However, a person skilled in the art should understand that these descriptions are exemplary and do not limit the protection scope of the present disclosure. Moreover, a person skilled in the art can make various variations and modifications to the present disclosure according to the spirit and principles of the present disclosure, and these variations and modifications also fall within the scope of the present disclosure.

What is claimed is:

1. A moat-type microwell culture plate, comprising a microwell culture plate body (1), wherein the microwell culture plate body (1) is provided with a water injection groove (2); and micro culture wells (3) are arranged inside the water injection groove (2), with openings of the culture wells facing upward.

2. The moat-type microwell culture plate according to claim 1, wherein the water injection groove (2) and the culture wells are of an integrated structure.

3. The moat-type microwell culture plate according to claim 1, wherein the culture wells arranged inside the water injection groove (2) have equal transverse and longitudinal spacings, and both the transverse and longitudinal spacings between the culture wells are 9 mm.

4. The moat-type microwell culture plate according to claim 1, wherein the microwell culture plate body (1) is surrounded by a rim (5); an annular groove (4) is formed at a bottom of the rim (5); an inner edge of the annular groove (4) is integrally connected to the microwell culture plate body (1); and a reinforcing rib connected to the microwell culture plate body is disposed in the annular groove (4).
